(19) United States
(12) Patent Application Publication
(10) Pub. No.: US 2026/0120867 A1
(43) Pub. Date: Apr. 30, 2026

(54) SYSTEMS AND METHODS FOR FUNCTIONAL LUMEN IMAGING PROBE DIAGNOSTIC ENVIRONMENT

(71) Applicant: Northwestern Memorial Healthcare, Chicago, IL (US)

(72) Inventors: John Erik PANDOLFINO, Wilmette, IL (US); Sourav HALDER, Chicago, IL (US); Neelesh A. PATANKAR, Evanston, IL (US); Wenjun KOU, Houston, TX (US); Dustin Allan CARLSON, Chicago, IL (US)

(21) Appl. No.: 18/932,941

(22) Filed: Oct. 31, 2024

(51) Int. Cl. G16H 50/20; G16H 30/40

(52) U.S. Cl. CPC G16H 50/20; G16H 10/60; G16H 20/00; G16H 30/40

(57) ABSTRACT

Systems and methods include a diagnostic evaluation environment that may provide probabilities of occurrence for one or more conditions based on evaluation of an input dataset. The probabilities may be based on multivariate evaluation of the input dataset against one or more pre-determined clinical metrics. In operation, a distributed environment may process the input dataset to visualize information for a given range and then provide probabilities and associated metric values for different occurrences and conditions to provide diagnostic support to treat one or more conditions.

### GOVERNMENT LICENSE RIGHTS

This invention was made with government support under RO1-DK079902 and P01-DK117824 awarded by the National Institute of Diabetes and Digestive and Kidney Diseases (NIDDK). The government has certain rights in the invention.

### BACKGROUND

### 1. Field of Disclosure

Embodiments of the present disclosure relate to systems and methods for diagnostic evaluation of medical data. Specifically, one or more embodiments are directed toward quantitative assessments of medical data to evaluate organ function and/or quality.

### 2. Description of Related Art

Various tools may be used to assess motility disorders for organs that may expand/contract and/or be analyzed based on different muscle functions, such as relaxation and contraction of one or more sphincters. One approach may include impedance planimetry to measure dimensions of a lumen inserted within an organ, such as by using a functional lumen imaging probe (FLIP). While approaches like FLIP can provide rich data, the data often lacks sufficient relationships to various organ functionality to provide true diagnostic information, and as a result, FLIP may be used as a complimentary tool to other approaches and/or may experience slow adoption.

### SUMMARY

Applicant recognized the problems noted above herein and conceived and developed embodiments of systems and methods, according to the present disclosure, for performing manometry procedures.

In an embodiment, a method includes receiving a data input including data acquired by one or more medical devices. The method also includes determining, based at least in part on one or more boundary conditions, a range within the data input. The method further includes generating one or more visualizations corresponding to the range. The method also includes determining, for one or more diagnostic conditions, respective probabilities of occurrence. The method includes determining, for the one or more diagnostic conditions, respective values corresponding to one or more pre-determined clinical metrics. The method also includes providing, for viewing on a display, at least one of the one or more visualizations, the respective probabilities of occurrence, or the one or more pre-determined clinical metrics.

In an embodiment, a processor includes one or more circuits to generate one or more visualizations for a medical device data input, the one or more visualizations corresponding to a region of a patient identified by an input from a user. The one or more circuits are also to determine, using one or more machine learning algorithms, one or more probabilities of occurrence for one or more diagnostic groups. The one or more circuits are further to determine one or more clinical metrics for the medical device data input. The one or more circuits are also to provide, on a display and responsive to a request, at least one of the one or more visualizations, the one or more probabilities of occurrence, or the one or more clinical metrics.

In an embodiment, a computer-implemented method includes generating one or more visualizations for a medical device data input, the one or more visualizations corresponding to a region of a patient identified by an input from a user. The method also includes determining, using one or more machine learning algorithms, one or more probabilities of occurrence for one or more diagnostic groups. The method further includes determining one or more clinical metrics for the medical device data input. The method includes providing, on a display and responsive to a request, at least one of the one or more visualizations, the one or more probabilities of occurrence, or the one or more clinical metrics.

### BRIEF DESCRIPTION OF DRAWINGS

The present technology will be better understood on reading the following detailed description of non-limiting embodiments thereof, and on examining the accompanying drawings, in which:

FIG. 1 illustrates an example system for a diagnostic evaluation environment, in accordance with embodiments of the present disclosure;

FIG. 2 illustrates an example representation of a virtual disease landscape, in accordance with embodiments of the present disclosure;

FIG. 3A illustrates an example representation of data visualizations, in accordance with embodiments of the present disclosure;

FIG. 3B illustrates an example representation of one or more metrics associated with the environment, in accordance with embodiments of the present disclosure;

FIG. 4A illustrates an example representation of an interface for a diagnostic environment, in accordance with embodiments of the present disclosure;

FIG. 4B illustrates an example representation of an interface for a diagnostic environment, in accordance with embodiments of the present disclosure;

FIG. 4C illustrates an example visualization of a range of data provided to the diagnostic environment, in accordance with embodiments of the present disclosure;

FIG. 4D illustrates an example representation of an interface for a diagnostic environment, in accordance with embodiments of the present disclosure;

FIG. 4E illustrates an example visualization of a range of data provided to the diagnostic environment, in accordance with embodiments of the present disclosure;

FIG. 4F illustrates an example visualization of a range of data provided to the diagnostic environment, in accordance with embodiments of the present disclosure;

FIGS. 4G-4I illustrate example representations of a virtual disease landscape, in accordance with embodiments of the present disclosure;

FIG. 4J illustrates an example representation of one or more metrics associated with the environment, in accordance with embodiments of the present disclosure;

FIG. 5 is a flow chart of a process for generating a data visualization, in accordance with embodiments of the present disclosure;

FIG. 6 is a flow chart of a process for evaluating input data for a diagnostic system, in accordance with embodiments of the present disclosure;

FIG. 7 is an example configuration for a computing device, in accordance with embodiments of the present disclosure.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose. Additionally, like reference numerals may be used for like components, but such use should not be interpreted as limiting the disclosure.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions. Like numbers may be used to refer to like elements throughout, but it should be appreciated that using like numbers is for convenience and clarity and not intended to limit embodiments of the present disclosure. Similarly, using different numbering does not necessarily imply that components are different or cannot share one or more features with differently numbered components. Moreover, references to “substantially” or “approximately” or “about” may refer to differences within ranges of +/−10 percent.

Embodiments of the present disclosure may be directed toward systems and methods for diagnostic support with respect to data acquired during one or more procedures, such as medical procedures. Various embodiments may be directed toward one or more evaluation environments that may be used to receive input data, such as raw and/or processed data from a medical procedure, and then provide diagnostic assistance based an analysis of the data compared to one or more metrics. In at least one embodiment, diagnostic assistance may include providing one or more probabilities for one or more conditions and/or causes, for example, by identifying a probability that data associated with a patent may be grouped within one or more categories. Different metrics may be weighted and/or correlated across a multi-dimensional latent space to provide a representation that may be used to sort or otherwise group data within different categories for a given subset of metrics. Combinations of metrics may be used to provide diagnostic support for a variety of different conditions and/or possible ailments for a reviewing physician to consider when providing a diagnostic opinion to a patient. Various embodiments address and overcome problems with existing systems that lack diagnostic support to accompany acquired data. Furthermore, one or more embodiments may be used to provide a unified platform for a multifaceted analysis to provide a wholistic evaluation over a number of different metrics, which may be used as diagnostic support and/or guidance for a reviewing physician.

Systems and methods may be used to leverage diagnostic data that may be acquired from one or more tools, such as a functional lumen imaging probe (FLIP). While FLIP has been used to, as one non-limiting example, acquire data to diagnose a variety of esophageal motility disorders, there is a lack of viable software for quantitative assessment of FLIP measurements. Embodiments address and overcome this problem by using a framework, which may be a web-based framework, for a unified assessment of FLIP measurements including a variety of different clinical metrics to provide a tool for diagnostic assistance. By way of example, systems and methods may identify and/or selectively use one or more clinical metrics to generate probabilities with respect to organ operation, function, and/or the like. As one non-limiting example related to esophageal motility disorders, metrics may include information such as esophagogastric junction (EGJ) distensibility index (DI), maximum EGJ opening diameter, and/or mechanics-based metrics for estimating strength and effectiveness of contractions such as contraction power and displaced volume. Furthermore, one or more embodiments may use various machine learning (ML)-based systems, such as clustering and predictive algorithms, to generate one or more predictions with respect to different conditions or disorders. At least one embodiment may deploy a virtual disease landscape (VDL) for visualization and/or classification of different input data. The VDL may represent the multi-dimensional latent space where processed data from the FLIP measurements may be positioned to determine a likelihood of different conditions, causes, and/or the like.

Embodiments of the present disclosure may further be used to increase adoption and/or use of FLIP, as opposed to or in additional to, other techniques such as high resolution manometry (HRM). Advantageously, FLIP may provide practitioners with improved ease of use as well as advantages to patients because FLIP may be performed while the patient is sedated, thereby providing an alternative to traditional approaches for obtaining medical data. Systems and methods may provide a standardized, unified platform to perform post-processing and/or a multifaceted analysis of FLIP data, such as diameter and pressure readings, against a number of clinical metrics. The clinical metrics may be based on physical properties of the organ, for example, physical movement or operation of the organ. The metrics may provide for a multi-variable analysis to extract and identify key findings from the FLIP data that may otherwise be unrelated when viewed individually. Accordingly, the collection of metrics within the platform may provide a unified estimate of the state and functioning of the organ (e.g., the esophagus) based on FLIP measurements.

Various embodiments of the present disclosure may be used as a diagnostic tool to provide estimates of probabilities for one or more categories or disorders and to provide measurements according to one or more metrics to improve diagnostic evaluations for different patients. In at least one embodiment, systems and methods may be used as a diagnostic tool to estimate both organ functionality and also physical properties that may be related to that functionality, thereby providing a meaningful way to diagnose and track treatment over time. For example, embodiments address and overcome problems with current systems that may provide a binary analysis of a set of information (e.g., healthy or unhealthy, normal or abnormal, etc.). Merely identifying a grouping does not provide the practitioner or patient with sufficient information to make informed decisions about diagnosis or treatment options, much less provide a metric by which treatment can be evaluated to determine whether or not treatment is working as expected and targeting the proper physical causes of the diagnosis. Embodiments address and overcome these problems, among others, by providing tools to evaluate and extract meaningful information from collected data and then to provide information that can be used to make an informed diagnostic and treatment decision. For example, systems and methods may identify a likelihood of one or more classifications, may identify how certain information compared to different metrics for physical characteristics of an organ, and provide visualized data so that a practitioner can analyze information to make a diagnostic decision, which may include a diagnostic recommendation provided by the framework and/or may be based, at least in part, on the probabilities and information provided.

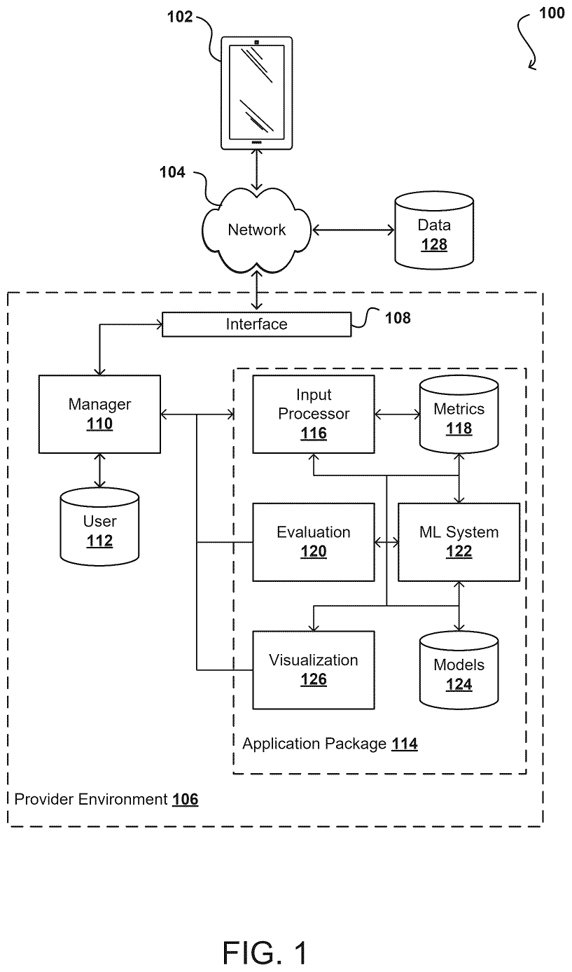

FIG. 1 illustrates an example system 100 that may be used with embodiments of the present disclosure. In this example, a computing device 102 (e.g., user device, compute device, client device, etc.) can submit a request over at least one network 104 to be received by a provider environment 106. The provider environment 106 may be an online platform or service environment provided by a service provider and/or for an affiliate, for example the environment 106 may be hosted or otherwise provided via one or more cloud resource providers on behalf of a service provider. The client computing device 102 may be a representative and/or act as a proxy for one or more users that may be submitting requests. For example, a user may navigate to one or more dashboards, web applications, landing pages, or access points using the device to submit a request, among other options. Additionally, in at least one embodiment, the client computing device 102 may act as a proxy to execute stored instructions to make and receive requests. For example, the client computing device 102 may send a request responsive to receiving one or more inputs and/or the like. As another example, a request may be transmitted as part of an automated or semi-automated workflow, which may or may not receive user interaction. Moreover, the computing device 102 may be a device transmitting data and/or measurement information to the provider environment 106, either directly as the device acquiring the measurements and/or as the device receiving and/or processing measurement data from the device. Accordingly, the client computing device 102 may be used with direct input from one or more users, from stored software instructions, from executions of various workflows, or combinations thereof.

In at least some embodiments, the request can include a request to execute one or more workflows associated with analysis and/or processing of electronic health records (EHR), including evaluation of data (e.g., imaging data, video data, text data, audio data, raw data from one or more medical devices, processed data from one or more medical devices, combinations thereof, etc.), among other options. In many cases, submitted requests may include both a request to access data (e.g., stored data, streaming data, etc.) and also a request to process the data using one or more workflows associated with the environment 106. However, embodiments may also be used with singular requests or portions of requests, as discussed herein. In at least one embodiment, a selected workflow may be based, at least in part, on information provided by the computing device 102, such as a command, or based on data received by the environment 106. The network(s) 104 can include any appropriate network, such as the Internet, a local area network (LAN), a cellular network, an Ethernet, or other such wired and/or wireless network. The provider environment 106 can include any appropriate resources for accessing data or information, such as EHR, as may include various servers, data stores, and other such components known or used for accessing data and/or processing data from across a network (or from the “cloud”). Moreover, the client computing device 102 can be any appropriate computing or processing device, as may include a desktop or notebook computer, smartphone, tablet, wearable computer (e.g., smart watch, glasses, contacts, headset, etc.), server, or other such system or device.

An interface layer 108, when receiving a request or call, can determine the type of call or request and cause information to be forwarded to the appropriate component or sub-system. For example, the interface 108 may be associated with one or more landing pages, as an example, to guide a user toward a workflow or action. In at least one embodiment, the interface layer 108 may include other functionality and implementations, such as load balancing and the like.

Various embodiments of the present disclosure are directed toward processing and/or evaluating EHR, among other potential use cases, and as a result certain data protection operations may be deployed. In at least one embodiment, a manager 110 may receive various calls and/or requests provided to the provider environment 106. The manager 110 may determine whether a requestor or provider is authorized to perform one or more tasks and/or to access certain data. For example, a user datastore 112 may include credential information for users associated with the devices 102 and/or authorized users acting on behalf of the devices 102. In this manner, sensitive information may be protected and access may be restricted to those with permission. Verification of users, for example via submitted credentials, may also determine a level of accessibility within the environment 106, which may be on an application-basis, a user-basis, or some combination thereof. For example, a first user may have access to the environment, but only have a limited set of applications that are accessible, while a second user may have access to more applications, and a third user may be entirely barred from the environment. Accordingly, access may be controlled and information related to EHR may be protected.

Systems and methods may include a web-based or application-based portal that permits receipt, analysis, and evaluation of information, such as EHR, that may include multi-modal information including text data, video data, image data, audio data, numerical data, data acquired from one or more signals generated by a device, and/or combinations thereof. For example, EHR may include data or information acquired during one or more procedures, such as during a procedure using FLIP where pressure and/or diameter information may be acquired, stored, and/or processed. The information may include combinations of information, such as pressure readings, which may be signals converted to a unit, diameter information, and/or combinations thereof. Furthermore, the information may be multi-modal, which may include physical notes or information along with the testing information.

An application package 114 may be executable within the environment 106 to provide functionality to the client device 102 according to one or more embodiments. In this example, the application package 114 may be associated with different sets of executable software instructions, which may share one or more underlying resources, to perform one or more tasks. The application package 114 may also be part of a service that includes multiple levels of use, where a user at a first level may have permission for a subset of application package capabilities while another user with a second level may have permission for a different subset of application package capabilities.

In this non-limiting example, the application package 114 includes an input processor 116 that may be used to process and/or format input information based, at least in part, on a target workflow. For example, one or more embodiments of the present disclosure may implement different processing techniques to standardize the information acquired from various medical procedures in order to provide a consistent approach to metric determination. Without a standard approach for pre-processing and/or analysis and data, the calculated metrics can be incorrect and lead to wrong diagnosis. Example approaches may include binning or otherwise clipping information, artifact removal, maximum/minimum intervals, volume conversation, and/or the like.

In at least one embodiment, the input processor 116 may perform task intermittently during evaluation of data using one or more portions of the application package 114. For example, an initial set of parameters may be used to format or otherwise prepare data for analysis and then the user may provide additional inputs at different points of data analysis in order to tune or otherwise direct the analysis. In this manner, systems and methods may be used to provide a consistent approach to metric evaluation while also permitting individual patient information as an input to drive data analysis to provide more accurate, personalized results.

Turning to the example for FLIP directed to an esophageal evaluation, pre-processing may include, eliminating certain readings due to physical constraints of the system. For the example of clinical EGJ metrics (which are estimated at 60 ml and 70 ml), the readings at the initial 5 seconds after filling to each volume may be eliminated from consideration to allow time for the esophagus to stabilize. As another example for esophageal evaluation, artifact identification and removal may address problems with a dry catheter artifact (DCA) and an EGJ movement artifact (EMA). DCA occurs when the FLIP bag is fully occluded, causing potentially incorrect measurements of abnormally high diameters distal to the occluded section. To avoid DCA, readings may be eliminated from consideration at one or more identified times, such as time instants (along with some buffer, such as ±0.1 s) when the minimum diameter at any point along the FLIP is less than or equal to a threshold, such as 6 mm, unless it is determined that the reading is acquired at the EGJ itself. The EGJ midline may be tracked by the minimum diameter between the proximal and distal limits of the EGJ, and may be set by the user as discussed herein. Often this midline may shift unnaturally due to noisy variations in diameter measurements leading to EDA. Embodiments of the present disclosure may deploy one or more thresholds for location stability. For example, a threshold of 0.5 seconds of stable EGJ midline location may be used for information to be configured for analysis. Various pre-processing may also be used for the mechanics-based metrics and the VDL, such as a volume conservation constraint to ensure the physical limitations of a fully enclosed system where fluid cannot escape.

As discussed herein, one or more metrics may be used to evaluate information acquired from one or more medical procedures, such as FLIP. The illustrated embodiment includes the metrics datastore 118 that may be used to store metrics and may be updated over time, for example based on continued evaluation and/or analysis using one or more ML systems, in order to provide evaluation criteria for the evaluation engine 120. For example, the metrics datastore 118 may be used to determine which information is considered for evaluation, such as via the input processor 116, to determine how to present data to a user, and/or combinations thereof. Furthermore, the metric datastore 118 may provide one or more inputs to the evaluation engine 120 and/or one or more ML systems 122. The ML systems 122 may include one or more trained models that may receive, as an input, at least a portion of the data acquired from the medical procedure, such as FLIP data. The data may be processed using one or more trained models to provide a prediction or probability related to one or more outcomes, such as a determination regarding a probability of a disorder according to one or more classifications. For example, a probability over a certain quantity may lead to assignment to a given classification. As another example, a determination that a set of data is associated with a given classification may be used to assign a probability or other information that may be used by a physician to provide a diagnosis.

The ML system 122 may execute using one or more trained models, which may be stored in a model datastore 124 and may be accessible during operation of the application package 114. For example, a particular model may be selected based on the type of analysis being requested. Additionally, models may be fine-tuned or particularized for different use cases, such as a model for esophageal evaluation, a model for heart evaluation, and/or the like. As discussed herein, models may be periodically updated or retrained as new information is acquired, such as output information generated by the models that is validated by one or more clinicians, among other examples. In at least one embodiment, systems and methods of the present disclosure deploy the one or more ML systems 122, which may include execution of different software instructions based, at least in part, on a request received from the user device. The software instructions may execute on local or remote hardware, for example using one or more distributed compute services. The models associated with the model datastore 124 may be trained and or execute using one or more different sets of parameters, which may be associated with various training runs that were used to develop weights for different layers of the model. The models 124 may execute using different settings, hyperparameters, rules, and/or the like that may be tuned based on one or more use cases. When developing and training the models, the training data may be labeled or unlabeled and/or may be augmented or otherwise influenced by one or more human reviewers, but it should be appreciated that raw training data may be used with one or more self-supervised learning processes. In one embodiment, training data may include information that is validated by one or more expert clinicians and then model outputs may also be evaluated and validated as part of the training process to adjust different weights for the models.

In at least some embodiments, language models, such as large language models (LLMs) or visional language models (VLMs) and/or other types of generative artificial intelligence (AI) may be implemented as part of the ML system 122. These models may be capable of understanding, summarizing, translating, and/or otherwise generating text (e.g., natural language text, labels, etc.), images, video, and/or the like, based on the context provided in input prompts or queries. The models (e.g., LLMs, VLMs, etc.) may be used for summarizing textual data, analyzing and extracting insights from data (e.g., textual, image, video, etc.), and generating new content (e.g., text, image, video, audio, etc.). Various embodiments may also include single modality models (e.g., exclusively for text or image processing) or multi-modality models (e.g., receiving combinations of inputs). For example, VLMs may accept image, video, audio, textual, 3D design, and/or other inputs data types and/or generate or output image, video, audio, textual, 3D design, and/or other output data types.

Various types of architectures may be implemented in various embodiments, and in certain embodiments, architecture may be technique-specific. As one example, architectures may include recurrent neural networks (RNNs) or long short-term memory networks (LSTMs), transformer architectures (e.g., self-attention mechanisms, encoder and/or decoder blocks, etc.), convolutional neural networks (CNNs), and/or the like. Furthermore, various models may be trained to execute using a variety of different methods, such as Bayesian statistical inference, among others.

At least one embodiment of the present disclosure may include one or more neural networks configured to Bayesian inference to learn a probability distribution. Implementing Bayesian statistical inference may address and overcome problems with overfitting, which may occur when a model optimizes its objective function based on a large set of training data. Bayesian networks may model uncertainty within the different weights of the neural network. As will be appreciated, in Bayesian inference Bayes' rule is used to compute the posterior given a set of training data. Accordingly, one or more models may be used to specify distributions over model parameters and predictions with a quantified uncertainty. Smaller datasets may be used with a reduced risk of overfitting compared to standard neural networks.

In various embodiments, the models may be trained using unsupervised learning, in which models learn from large amounts of unlabeled training data. Furthermore, one or more models may be task-specific or domain-specific, which may be based on the type of training data used. Additionally, foundational models may be used and then tuned for specific tasks or domains. Some types of foundational models may include question-answering, summarization, filling in missing information, and translation. Additionally, specific models may also be used and/or augmented for certain tasks, using techniques like prompt tuning, fine-tuning, retrieval augmented generation (RAG), adding adapters, and/or, the like.

Systems and methods may also provide for a visualization engine 126 to provide an output to the device 102 responsive to the request. For example, embodiments may provide graphical representations of the information presented to the evaluation engine 120 and/or may plot or otherwise indicate a position of a given analysis within the VDL. Accordingly, systems and methods may be used to provide diagnostic assistance to a physician by providing a multivariate approach to identifying one or more probabilities with respect to a number of different potential diagnostic outcomes. In certain embodiments, systems and methods may aggregate or otherwise provide a final prediction and/or recommendation. However, in other embodiments, systems and methods may provide probabilities and visualization to guide physicians toward developing their own diagnosis.

Various embodiments may receive data for evaluation from one or more datastores 128. The datastores 128 may part of the same provider environment 106 and/or may be hosted by another provider or locally with the device 102. Furthermore, data may be streamed directly to the environment 106 for analysis in real or near-real time.

One or more embodiments may be used with FLIP, but it should be appreciated that embodiments are not limited to information collected by such a tool and that various embodiments of the present disclosure may be used with a variety of data, which may include data collected during one or more medical procedures. Turning to the example of FLIP, the tool generally includes a catheter with 16 impedance planimetry sensors, which may be spaced along a length or a portion of the length. As one example, the sensors may be spaced along a subset of the length at 1 cm intervals. The tool may also include a pressure sensor at a distal end. The catheter may be mounted with an inflatable bag. In operation, the testing protocol involves placing the FLIP at the EGJ with approximately 2-3 cm from its distal end and incrementally distending the bag with saline to various volumes (e.g., 40 ml-70 ml) in steps of 10 ml. For example, one procedure may include distension at 40 ml, then 50 ml, then 60 ml, and then 70 ml. Furthermore, the volumes may be held for approximately 60 seconds. Due to the distension of the bag, a secondary peristaltic contractile response called repetitive antegrade contractions (RACs) is induced in normal motility subjects. Based on the contractile response and the nature of EGJ opening during distension at 60 and 70 ml, patients may be diagnosed into different categories.

Various embodiments may use one or more clinical metrics for diagnosing a variety of disorders. As one example with esophageal motility disorders, two categories of disorders may include contractile response in the esophageal body and the nature of EGJ opening. Furthermore, each of these categories of disorders may be further categorized by sub-metrics. By way of example, the contractile response may be categorized into normal, borderline, impaired/disordered, absent, and spastic-reactive, based on the contraction pattern. However, the EGJ opening may be quantified through two metrics: EGJ distensibility index (EGJ-DI) and maximum EGJ opening diameter, which may be used for categorization into a different set of groups, such as: normal EGJ opening (NEO), borderline-normal EGJ opening (BNEO), borderline-reduced EGJ opening (BREO), and reduced EGJ opening (REO). Systems and methods of the present disclosure have developed and implement mechanics-based metrics to quantify the strength and effectiveness of contractions using contraction power (CP) and displaced volume (DV). Furthermore, embodiments have also developed and implemented ML-based metrics. For example, one or more embodiments may be used to determine a probability of obstruction at EGJ using one or more trained models. Additionally, as discussed herein, systems and methods may develop and implement one or more clustering applications in order to cluster various esophageal motility disorders in a three-dimensional (3D) latent space, which may be referred to as the VDL, to assign a probability of motility among four groups: normal, weak, obstruction, and spastic-reactive. In this manner, systems and methods address and overcome problems associated with limited readability and implement of FLIP data by incorporating a variety of techniques to develop a deeper understanding and classification of acquired data.

Systems and methods of the present disclosure may be directed toward an automated and/or semi-automated program to predict one or more metrics associated with one or more target diagnostic evaluations. One or more embodiments may enable fine-tuning and/or interaction with one or more users in order to target or otherwise provide more meaningful control and/or analysis of various output predictions. By providing users with additional insight and control into the process, thereby reducing risks of misdiagnosis and/or misunderstanding compared to systems that merely generate data without permitting the user to see how different formulations and analysis are performed. However, substantial input requirements may cause users to resist use of the system, and therefore, embodiments may target and tune the required inputs for a given user and/or diagnostic based on one or more factors that may be relevant to the analysis. For example, one or more inputs may be deemed salient, such as a specification of bounds when evaluating FLIP data. This information may be important for a number of reasons, for example, because the EGJ location can vary from patient to patient and/or the location may move due to certain conditions (e.g., spastic reactive cases), thereby reducing opportunities to automate the system and/or reducing errors from attempts to guess or otherwise statistically determine the location. Similarly, the user may provide additional thresholding bounds for noise measurements and/or evaluation to tune the operation of the evaluation engine. For example, a user may provide one or more manual inputs for a “peak” associated with an EGJ opening diameter. Systems and methods may provide an interaction environment to receive inputs from the user, such as through peripheral devices (e.g., mouse, touchscreen etc.), audio, video, text, and/or combinations thereof. In this manner, systems and methods of the present disclosure may provide a framework to analyze input data from one or more medical procedures to predict important clinical, mechanical, and ML-based metrics in a semi-automatic manner.

Embodiments of the present disclosure may be used to address and overcome problems with existing techniques and/or to address gaps in existing technologies with respect to use and evaluation of data, such as from medical devices. In the non-limiting example of FLIP, embodiments provide an integrated analysis platform that uses a variety of different metrics. Unlike other techniques, such as HRM, metrics for FLIP may be lacking and/or insufficient to provide full diagnostic assistance. Embodiments of the present disclosure may incorporate a variety of different metrics for evaluation to predict probabilities for different classifications and/or groupings. By way of example, systems and methods may include metrics for integrated relaxation pressures (IRP) (e.g., EGJ-DI, max diameter, obstruction probability, etc.), peristalsis, delayed cerebral ischemia (DCI) (e.g., power and work), latency, morphology, positive end-expiratory pressure (e.g., intrabag pressure), and/or the like. Systems and methods may therefore be used to improve classifications while also supporting machine learning implementations through the VDL.

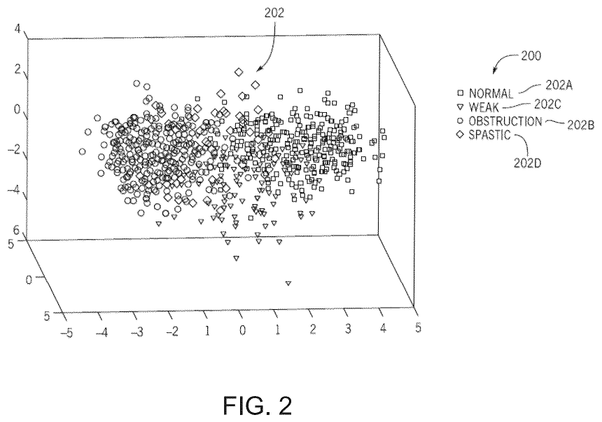

FIG. 2 illustrates an example representation for a virtual disease landscape (VDL) 200 that may be used with embodiments of the present disclosure. Visualization of information may be one part of the framework of an ensemble of various mechanics-based and deep learning techniques that may qualify a “health” or “score” for a given organ of a patient, which in this example may be an esophagus. Embodiments of the present disclosure may be used to estimate an effectiveness of treatment and/or to provide diagnostic evaluations, among other options. Systems and methods may include focused visualization of different measurements for diagnosis, mechanics-based metrics to quantify function of the organ, and/or the VDL to quantify the distinct nature of various disorders. The illustrated VDL 200 is provided by way of example and in at least one embodiment, systems and methods may develop different landscapes having more or less than three dimensions. For example, the VDL may be represented by an X-dimension latent space, but may be mapped to a lower-dimensional space for the purposes of visualization, for example using a variational autoencoder.

In this example, the VDL 200 may be representative of a variety of different disorders or classifications 202 that may be clustered within the space. As shown, several distinct clusters or classifications may be present in the VDL 200. For example, a first cluster 202A corresponding to normal operation is shown with minimal overlap with a second cluster 202B corresponding to an obstruction. Additional clusters in this example include a third cluster 202C indicative of a weak classification and a fourth cluster 202D indicative of spastic classification. Data provided by a user may be evaluated and then plotted within the VDL 200 to provide a visual indication to the user regarding probabilities that the information associated with the data is grouped within one or more clusters 202. In this manner, a physician may receive information that may help information or otherwise guide diagnostic decisions.

However, as shown in the example VDL 200, there may not be a clean delineation between the different clusters 202. For example, with respect to the horizontal axis of the space, certain points associated with the third cluster 202B are intermixed with the first cluster 202A such that a linear divide could not be formed between the first cluster 202A and the third cluster 202C. Similarly, there are certain points associated with the first cluster 202A that are mixed into the second cluster 202B. Systems and methods of the present disclosure may address and overcome these deficiencies by using one or more algorithms to plot and/or classify new datapoints within the VDL 200 based, for example, on one or more metrics or parameters. For example, a K-nearest neighbor algorithm may be used to determine a closest number of points to a given new datapoint plotted within the VDL 200 to assign a classification and/or a probability associated with a classification. In this manner, a reviewing practitioner may receive information to help inform diagnostic decision making. In another example, systems and methods may generate a diagnostic and/or recommended diagnostic result based on the one or more algorithms. Furthermore, generation of the result may include identification of other information associated with the diagnostic classification, such as a recommendation for treatment options.

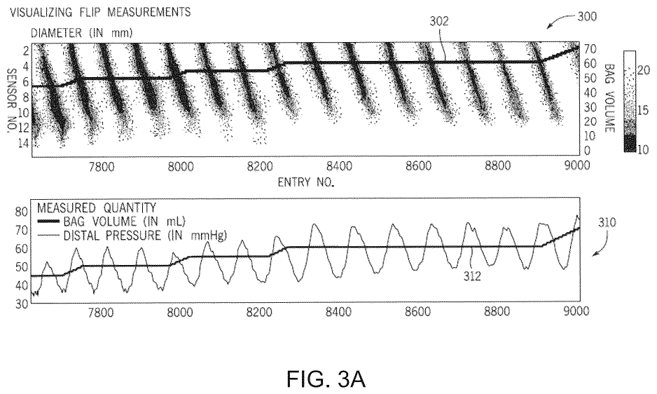

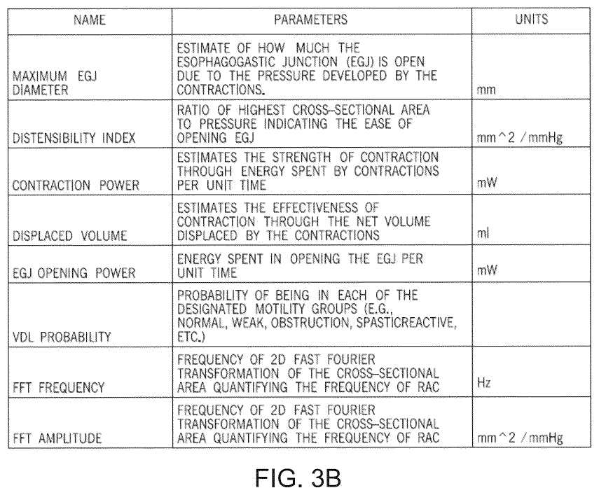

FIG. 3A illustrates representations 300, 310 that may be used to visualize data provided to the framework, which in this example is FLIP data associated with diameter in the representation 300 and bag volume/distal pressure in the representation 310. As shown with the representation 300, a reviewer may quickly determine that the input data was accurately (within some threshold) analyzed due to the expected behavior to be observed during the data gathering procedure. In these representations, the variations of diameter and pressure within a normal subject are illustrated. For example, measurement begins at approximately 50 ml and increases to 70 ml in a stepwise manner (as illustrated by the lines 302, 312 corresponding to bag volume). Furthermore, peristalsis due to the action of inflating the balloon is illustrated in the representation 310, as the pressure increases and decreases responsive to expansion and contraction of the esophagus. Such a condition may be expected during normal operation of the esophagus, potentially providing a datapoint to support a diagnosis from the clinician.

As discussed herein, embodiments may be used to establish one or more metrics for evaluation of different conditions and/or disorders and then systems and methods may determine probabilities or classifications based, at least in part, on the one or more metrics. Returning to the example of esophageal evaluation, metrics may include one or more of the metrics illustrated in FIG. 3B.

Systems and methods of the present disclosure may incorporate, within the framework, both visualization techniques discussed herein and also one or more algorithms to provide a decision support tool. Embodiments may use, as examples, one or both of a Bayesian plot of obstruction and the VDL to provide support in interpreting and evaluating acquired data in order to classify or otherwise diagnose an associated patient. To support diagnostics, one or more embodiments may be configured to output a probability with respect to one or more classifications along with one or more visual indicators or a description associated with data evaluation.

One or more embodiments may deploy a Bayesian approach to provide a diagnostic support tool that relies on a variety of different metrics to provide probabilities for different classifications or conditions. Various metrics may be correlated to different physical operations of a given organ in order to determine whether different sensor data correlates to a given classification with some probability. Returning to the example of the esophagus, an obstruction of a sphincter function may be associated with a pressure at a lower sphincter, but merely knowing the pressure does not provide the mechanics of that sphincter. Embodiments of the present disclosure, however, evaluate pressure along with changes in diameter, as one example, to determine the mechanical causes of the pressure changes to determine whether an obstruction is the problem, as opposed to some other cause. Embodiments may build one or more models or representations, such as the VDL, to cluster or otherwise facilitate identification of different classifications based on acquired sensor data. For example, at least one embodiment may incorporate K-nearest neighbor algorithms (where K may be a tunable hyperparameter) to determine an overall probability for a given diagnosis. In at least one embodiment, K-nearest neighbor algorithms may refer to one or more supervised learning methods used to classify and regress data points based on their proximity to other data points. That is, one or more algorithms may be used to assume that similar objects are in close proximity to one another when positioned within a latent space. As a result, “normal” functioning points should be close to other normal points. By mapping different points, based on information acquired from testing as dimensions of the vector, systems and methods may use the VDL to provide an initial classification of one or more new datapoints. Accordingly, systems and methods may be used to provide more information to a given set of sensor data by determining whether the sensor readings reasonably correlate to the mechanical structure of the organ.

Systems and methods may also use one or more metrics to evaluate success of treatment. For example, if a person is diagnosed as having an EGJ obstruction and the mechanical cause is shown to be the diameter, upon retesting after treatment, the diameter may be used as an indicator of treatment success or failure because, if the diameter were the cause of the condition, then treatment to increase the diameter would decrease the probability of the obstruction. Furthermore, the metrics may be used with further discussion and analysis, for example, with colleagues or to provide information to patients to explain why certain treatments and/or tests are necessary.

Embodiments may also use the metrics to enable a deterministic approach when developing different treatment options. For example, upon review of input information, it may be determined that a patient is normal with a first probability (e.g., 54%) and obstructed with a second probability (e.g., 48%). Based on these classifications, the metrics may be used to identify which treatment path may be more likely to improve the patient's condition. For example, if diameters for the patient are normal with a high probability, then surgical options may be eliminated as a treatment. Similarly, if the peristalsis also has a high metric for being normal, additional causes may be ruled out. The platform may provide real or near-real time determinations, for example during a procedure, which may permit practitioners to make additional evaluative decisions while the patient is in the room, for example, to collect additional information or to begin treatment for severe conditions. In this manner, analysis and presentation of the diagnostic support tool may be used to make decisions and alter treatment or evaluation procedures.

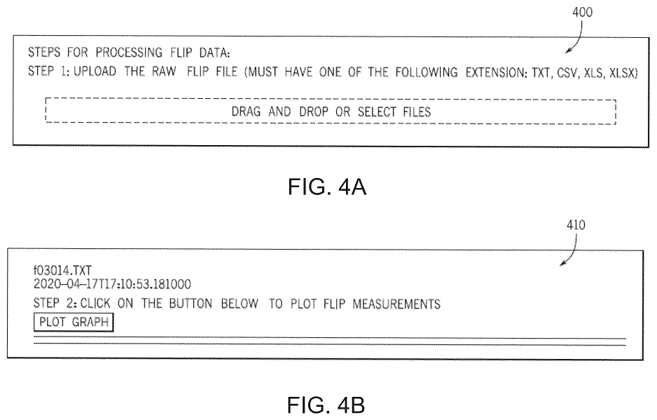

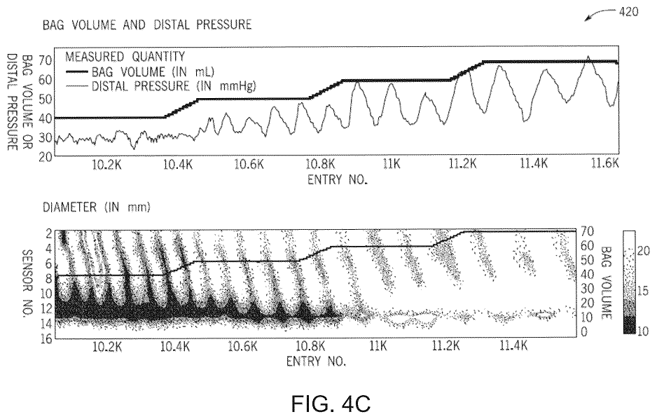

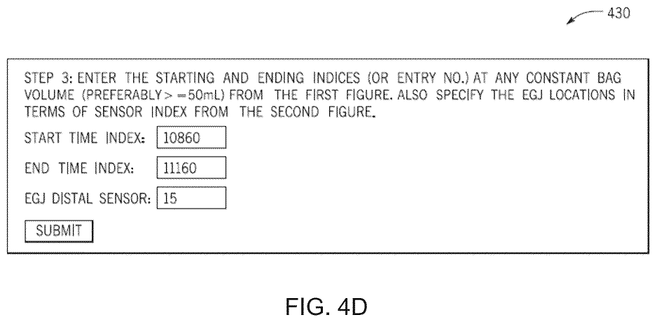

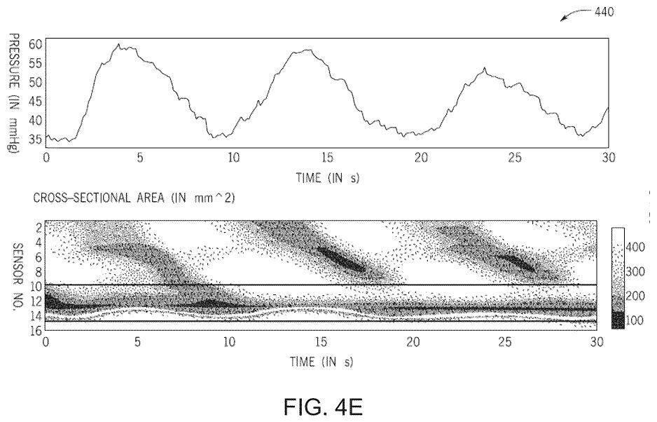

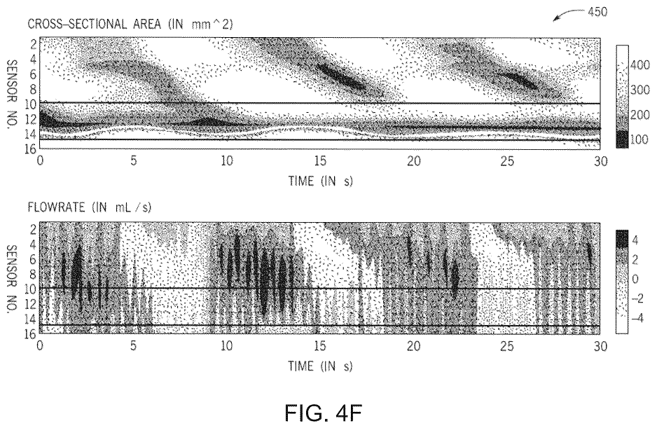

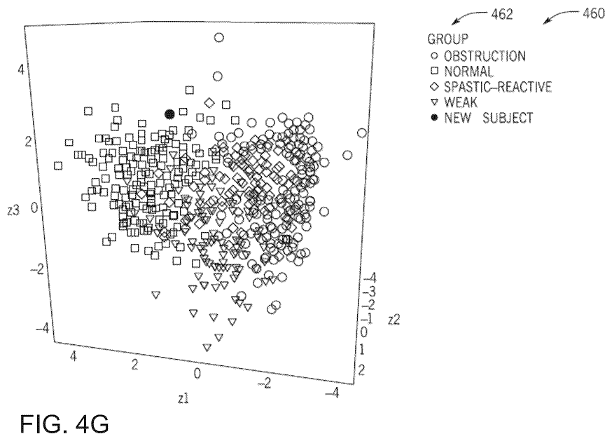

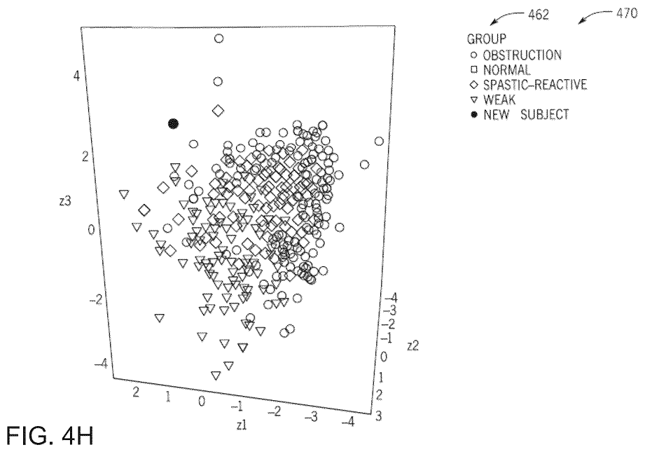

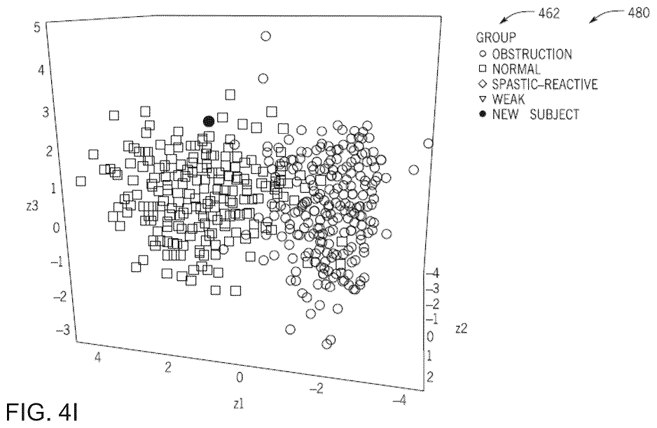

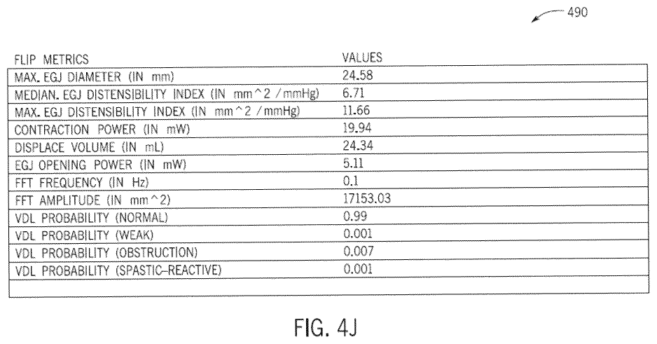

FIGS. 4A-4J illustrate example representations 400, 410, 420, 430, 440, 450, 460, 470, 480, 490 of interfaces that may be used with embodiments of the present disclosure. FIG. 4A illustrates the representation 400 that includes an option and instruction for the user to provide the data for evaluation to the service. As discussed herein, users may provide raw data that is evaluated, which may include pre-processing steps, by the platform. As shown, a variety of different types of data may be presented and used by the platform to extract salient information for analysis.

FIG. 4B illustrates the representation 410 illustrating the received file and also a request for the user to interact with the system in the form of a clickable icon. In at least one embodiment, systems and methods may include a semi-automated service that plots relevant information upon receipt of the appropriate file. However, in certain embodiments, the user may provide additional preferences for data visualization.

FIG. 4C illustrates the representation 420 including sample visualizations of different data acquired by the diagnostic device, which in this example includes a plot of bag volume and distal pressure and a heat map with respect to diameter.

FIG. 4D illustrates the representation 430 including a request for the user to provide an input. In this example, the request is associated with the EGJ location because, as noted herein, the EGJ location may be different for each patient. The illustrated embodiment correlate the location of the EGJ with a sensor associated with the tool. As a result, the user may provide information to the system to provide more detailed and accurate analysis.

FIGS. 4E and 4F illustrate representations 440, 450 of diagnostic results that may be generated by the system. In this example, the results are visualized and provided to the user for further analysis and evaluation. This example illustrates a rise and fall of pressure along with a cross-sectional area. As shown, as pressure rises, area increases between sensors 10-14. Presenting the data in such a way may enable practitioners to quickly identify errors within the analysis and/or guide diagnostic decisions.

FIGS. 4G-4J illustrate representations 460, 470, 480, 490 of diagnostic results that may be generated and used as a diagnostic tool. In this example, FIG. 4G illustrates a VDL representation 460 that includes different categories 462 within a 3D space and a target patient 464 plotted within the space. FIG. 4H provides additional clarity within the analysis, for example as a selectable option, by eliminating one or more clusters. Clusters may be eliminated based on low probabilities, high probabilities, target evaluations, and/or combinations thereof. For example, the category 462 associated with normal is removed from the representation 470 in FIG. 4H. Similarly, the categories 462 associated with spastic-reactive and weak are eliminated from FIG. 4I. In this manner, practitioners may visualize the data as a compact variable to inform their diagnostic decisions. Further diagnostic value may also be gleaned from the representation 490 of the various metrics used in the analysis, as shown in FIG. 4J.

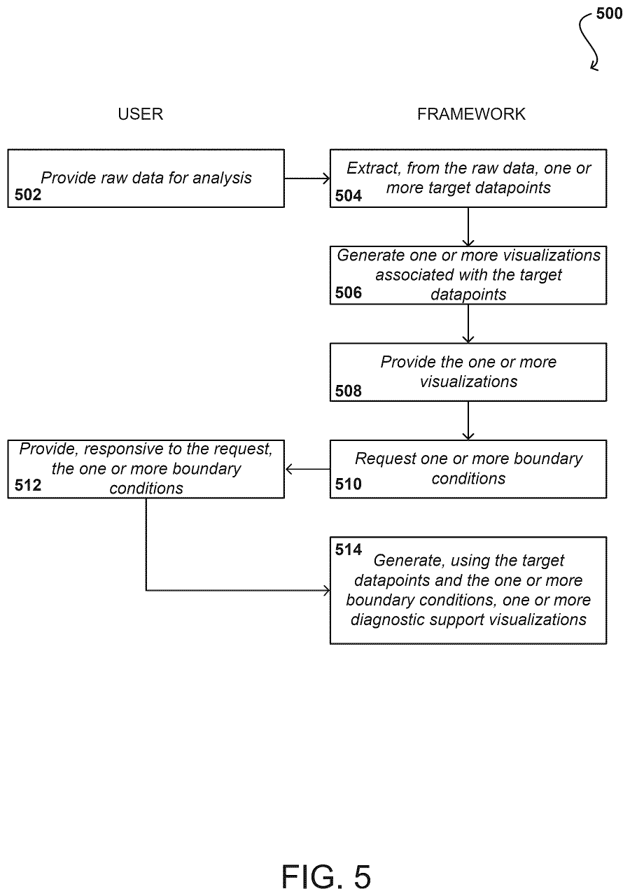

FIG. 5 illustrates an example flow chart in the form of a swimlane diagram for a process 500 for generating one or more diagnostic support visualizations. It should be appreciated that steps for the method may be performed in any order, or in parallel, unless otherwise specifically stated. Moreover, the method may include more or fewer steps. In this example, a left side is associated with operations by a user, which may be a human user or a device executing code responsive to one or more instructions, and the right side is associated with a framework, such as a web-based applications. The user may provide raw data for analysis 502. For example, the raw data may include data acquired during one or more diagnostic procedures, such as a medical procedure. It should be appreciated that embodiments are not limited to raw data and one or more pre-processing steps may be performed prior to uploading the data. However, as discussed herein, pre-processing may be performed by the framework for consistency across different data types.

In at least one embodiment, one or more target datapoints may be extracted 504. For example, data associated with particular metrics may be identified and extracted from the raw data. Furthermore, only subsets of the data may be used, such as data within a particular range or after a certain period of time. As discussed herein, the target datapoints may also include all or substantially all data that is later processed according to one or more parameters.

One or more visualizations may be generated 506. For example, a graphical representation of at least a portion of the data may be generated and provided to the user for review 508. Additionally, one or more boundary conditions may be requested 510. For example, the user may be requested to provide different minimum/maximum or identify a salient range, among other options. The user may then provide the one or more boundary conditions 512, which may be used to generate one or more diagnostic support visualizations.

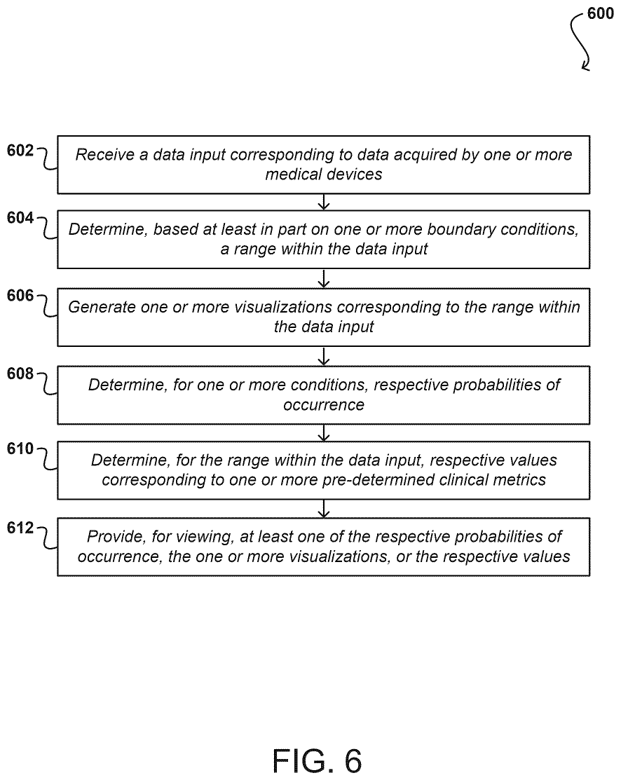

FIG. 6 illustrates an example flow chart for a process 600 for generating content that may support diagnostic processes. In this example, a data input is received 602. The data input may correspond to data acquired by one or more medical devices. For example, FLIP data may be received. In at least one embodiment, the data input may be raw data. One or more boundary conditions may be used to determine a range within the data input 604. The boundary conditions may be based on parameters of the evaluation, a user input, an evaluation of the data, and/or combinations thereof. In at least one embodiment, one or more visualizations may be generated for the data input 606. The one or more visualizations may be generated for a range within the data input, which may be the range associated with the boundary conditions. Additionally, in other embodiments, the range may be inferred based on the information extracted and evaluated.

In at least one embodiment, respective probabilities for one or more conditions of occurrence may be determined 608. For example, one or more features may be compressed and mapped within the VDL to determine a likelihood of occurrence based on an evaluation of other points in the VDL, such as using K-nearest neighbor or other similarity metrics. In at least one embodiment, one or more neural networks may be used to analyze and/or extract information from the data input. Additionally, in one or more embodiments, respective values corresponding to clinical metrics may be determined 610. Thereafter, at least one of the metrics, the probabilities, or the visualizations may be provided to a practitioner for review, which may aid or otherwise be used for diagnostic or treatment evaluation purposes.

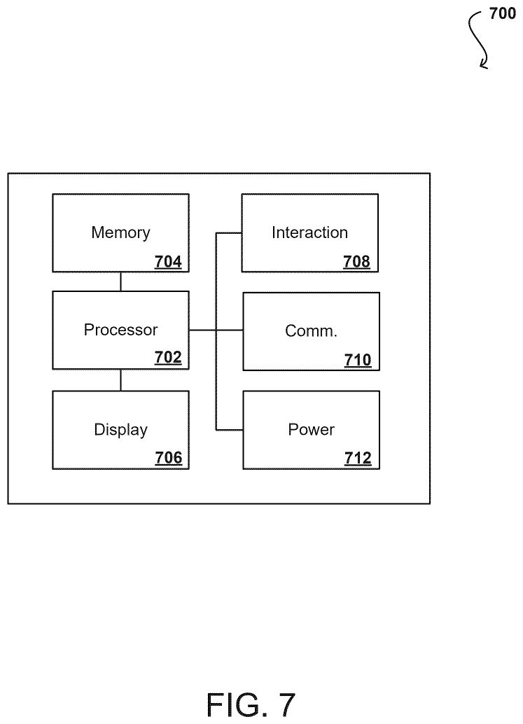

FIG. 7 illustrates a set of general components of an example computing device 700. In this example, the device includes a processor 702 for executing instructions that can be stored in a memory 704. The device can include many types of memory, data storage, or non-transitory computer-readable storage media, such as a first data storage for program instructions for execution by the processor 702, a separate storage for images or data, a removable memory for sharing information with other devices, etc. The device may optionally include a display element 706, such as a touch screen or liquid crystal display (LCD), although devices such as portable media players might convey information via other means, such as through audio speakers, and other devices may not include displays, such as server components executing within data centers, among other options. As discussed, the device in many embodiments will include at least one interaction component 708 able to receive input from a user. This input can include, for example, a push button, touch pad, touch screen, wheel, joystick, keyboard, mouse, keypad, or any other such device or element whereby a user can input a command to the device. In some embodiments, however, such a device might not include any buttons at all, and might be controlled only through a combination of visual and audio commands, such that a user can control the device without having to be in contact with the device. In some embodiments, the computing device 700 of FIG. 7 can include one or more network interface or communication components 710 for communicating over various networks, such as a Wi-Fi, Bluetooth, RF, wired, or wireless communication systems. The device may be configured to communicate with a network, such as the Internet, and may be able to communicate with other such devices. The device will also include one or more power components 712, such as power cords, power ports, batteries, wirelessly powered or rechargeable receivers, and the like.

Storage media and other non-transitory computer readable media for containing code, or portions of code, can include any appropriate media known or used in the art, including storage media and communication media, such as but not limited to volatile and non-volatile, removable and non-removable media implemented in any method or technology for storage of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

Embodiments may also be described in view of the following clauses:
1. A method, comprising:
receiving a data input including data acquired by one or more medical devices;
determining, based at least in part on one or more boundary conditions, a range within the data input;
generating one or more visualizations corresponding to the range;
determining, for one or more diagnostic conditions, respective probabilities of occurrence;
determining, for the one or more diagnostic conditions, respective values corresponding to one or more pre-determined clinical metrics; and
providing, for viewing on a display, at least one of the one or more visualizations, the respective probabilities of occurrence, or the one or more pre-determined clinical metrics.
2. The method of clause 1, wherein the one or more virtualizations include a virtual disease landscape representing a multi-dimensional latent space in a three-dimensional environment.
3. The method of clause 2, wherein the virtual disease landscape includes one or more regions associated with a diagnostic condition of the one or more diagnostic conditions.
4. The method of clause 3, further comprising:
receiving a second data input including second data acquired by the one or more medial devices;
determining a vector representation for the second data; and
plotting the vector representation in the virtual disease landscape.
5. The method of clause 1, wherein the respective probabilities of occurrence are computed using one or more K-nearest neighbor algorithms.
6. The method of clause 1, further comprising:
receiving, as an input provided at a visualization of the one or more visualizations, one or more evaluation parameters.
7. The method of clause 6, wherein the one or more medical devices is a functional lumen imaging probe and the one or more evaluation parameters includes a sensor location corresponding to an esophagogastric junction.
8. The method of clause 1, wherein the one or more pre-determined clinical metrics are determined by human reviewers.
9. The method of clause 1, further comprising
providing, based on at least one of the one or more visualizations, the respective probabilities of occurrence, or the one or more pre-determined clinical metrics, a diagnostic recommendation.
10. A processor, comprising:
one or more circuits to:
generate one or more visualizations for a medical device data input, the one or more visualizations corresponding to a region of a patient identified by an input from a user;
determine, using one or more machine learning algorithms, one or more probabilities of occurrence for one or more diagnostic groups;
determine one or more clinical metrics for the medical device data input; and
provide, on a display and responsive to a request, at least one of the one or more visualizations, the one or more probabilities of occurrence, or the one or more clinical metrics.
11. The processor of clause 10, wherein the one or more visualizations correspond to a multi-dimensional latent space representation presented as a three-dimensional environment.
12. The processor of clause 11, wherein the multi-dimensional latent space representation includes a grouped set of datapoints from historical data corresponding to the one or more diagnostic groups.
13. The processor of clause 10, wherein the one or more circuits are further to:
receive a second medical device data input;
determine, using at least a portion of the second medical device data input, a representation for display within the three-dimensional environment; and
providing the three-dimensional environment, with the representation, on the display.
14. The processor of clause 10, wherein the one or more probabilities of occurrence are computed using one or more K-nearest neighbor algorithms.
15. The processor of clause 10, wherein the input includes identification information for a location associated with a device used to acquire the medical device data input.
16. A computer-implemented method, comprising:
generating one or more visualizations for a medical device data input, the one or more visualizations corresponding to a region of a patient identified by an input from a user;
determining, using one or more machine learning algorithms, one or more probabilities of occurrence for one or more diagnostic groups;
determining one or more clinical metrics for the medical device data input; and
providing, on a display and responsive to a request, at least one of the one or more visualizations, the one or more probabilities of occurrence, or the one or more clinical metrics.
17. The computer-implemented method of clause 16, wherein the one or more visualizations correspond to a multi-dimensional latent space representation presented as a three-dimensional environment.
18. The computer-implemented method of clause 17, wherein the multi-dimensional latent space representation includes a grouped set of datapoints from historical data corresponding to the one or more diagnostic groups.
19. The computer-implemented method of clause 16, wherein the one or more circuits are further to:
receive a second medical device data input;
determine, using at least a portion of the second medical device data input, a representation for display within the three-dimensional environment; and
providing the three-dimensional environment, with the representation, on the display.
20. The computer-implemented method of clause 16, wherein the one or more probabilities of occurrence are computed using one or more K-nearest neighbor algorithms.
21. A method, comprising:
receiving a data input including data acquired by one or more medical devices;
determining, based at least in part on one or more boundary conditions, a range within the data input;
generating one or more visualizations corresponding to the range;
determining, for one or more diagnostic conditions, respective probabilities of occurrence;
determining, for the one or more diagnostic conditions, respective values corresponding to one or more pre-determined clinical metrics; and
providing, for viewing on a display, at least one of the one or more visualizations, the respective probabilities of occurrence, or the one or more pre-determined clinical metrics.
22. The method of clause 21, wherein the one or more virtualizations include a virtual disease landscape representing a multi-dimensional latent space in a three-dimensional environment.
23. The method of clause 22, wherein the virtual disease landscape includes one or more regions associated with a diagnostic condition of the one or more diagnostic conditions.
24. The method of clause 23, further comprising:
receiving a second data input including second data acquired by the one or more medial devices;
determining a vector representation for the second data; and
plotting the vector representation in the virtual disease landscape.
25. The method of any of clauses 21-24, wherein the respective probabilities of occurrence are computed using one or more K-nearest neighbor algorithms.
26. The method of any of clauses 21-25, further comprising:
receiving, as an input provided at a visualization of the one or more visualizations, one or more evaluation parameters.
27. The method of clause 26, wherein the one or more medical devices is a functional lumen imaging probe and the one or more evaluation parameters includes a sensor location corresponding to an esophagogastric junction.
28. The method of any of clauses 21-27, wherein the one or more pre-determined clinical metrics are determined by human reviewers.
29. The method of any of clauses 21-28, further comprising
providing, based on at least one of the one or more visualizations, the respective probabilities of occurrence, or the one or more pre-determined clinical metrics, a diagnostic recommendation.
30. A processor, comprising:
one or more circuits to:
generate one or more visualizations for a medical device data input, the one or more visualizations corresponding to a region of a patient identified by an input from a user;
determine, using one or more machine learning algorithms, one or more probabilities of occurrence for one or more diagnostic groups;
determine one or more clinical metrics for the medical device data input; and
provide, on a display and responsive to a request, at least one of the one or more visualizations, the one or more probabilities of occurrence, or the one or more clinical metrics.
31. The processor of clause 30, wherein the one or more visualizations correspond to a multi-dimensional latent space representation presented as a three-dimensional environment.
32. The processor of clause 31, wherein the multi-dimensional latent space representation includes a grouped set of datapoints from historical data corresponding to the one or more diagnostic groups.
33. The processor of any of clauses 30-32, wherein the one or more circuits are further to:
receive a second medical device data input;
determine, using at least a portion of the second medical device data input, a representation for display within the three-dimensional environment; and
providing the three-dimensional environment, with the representation, on the display.
34. The processor of any of clauses 30-33, wherein the one or more probabilities of occurrence are computed using one or more K-nearest neighbor algorithms.
35. The processor of any of clauses 30-24, wherein the input includes identification information for a location associated with a device used to acquire the medical device data input.
36. A computer-implemented method, comprising:
generating one or more visualizations for a medical device data input, the one or more visualizations corresponding to a region of a patient identified by an input from a user;
determining, using one or more machine learning algorithms, one or more probabilities of occurrence for one or more diagnostic groups;
determining one or more clinical metrics for the medical device data input; and
providing, on a display and responsive to a request, at least one of the one or more visualizations, the one or more probabilities of occurrence, or the one or more clinical metrics.
37. The computer-implemented method of clause 36, wherein the one or more visualizations correspond to a multi-dimensional latent space representation presented as a three-dimensional environment.
38. The computer-implemented method of clause 37, wherein the multi-dimensional latent space representation includes a grouped set of datapoints from historical data corresponding to the one or more diagnostic groups.
39. The computer-implemented method of any of clauses 36-38, wherein the one or more circuits are further to:
receive a second medical device data input;
determine, using at least a portion of the second medical device data input, a representation for display within the three-dimensional environment; and
providing the three-dimensional environment, with the representation, on the display.
40. The computer-implemented method of any of clauses 36-39, wherein the one or more probabilities of occurrence are computed using one or more K-nearest neighbor algorithms.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

1-9. (canceled)

10. A processor, comprising:
one or more circuits to:
generate one or more visualizations for a medical device data input, the one or more visualizations corresponding to a region of a patient identified by an input from a user corresponding to target boundary conditions for the medical device data input, wherein the medical device data input is extracted from a raw file;
determine, using one or more machine learning algorithms, one or more probabilities of an esophageal obstruction for one or more diagnostic groups;
determine a first mechanics-based metric corresponding to contraction power and a second metric corresponding to a maximum opening diameter of an esophagogastric junction (EGJ);
provide, on a display and responsive to a request, the one or more visualizations, the one or more probabilities, the first mechanics-based metric, and the second metric, as part of a diagnostic support environment;
generate an indication of a mechanical cause associated with the esophageal obstruction;
determine, based on the indication, a treatment recommendation as part of a deterministic evaluation comparing the indication to one or more classifications for the mechanical cause; and
determine, responsive to a second medical data device input after treatment for the mechanical cause based on the treatment recommendation, an assessment of treatment success based, at least in part, on an updated first mechanics-based metric, an updated second metric, and the treatment success metric.

11. The processor of claim 10, wherein the one or more visualizations correspond to a multi-dimensional latent space representation presented as a three-dimensional environment.

12. The processor of claim 11, wherein the multi-dimensional latent space representation includes a grouped set of datapoints from historical data corresponding to the one or more diagnostic groups.

13. The processor of claim 11, wherein the one or more circuits are further to:
receive the second medical device data input;
determine, using at least a portion of the second medical device data input, a representation for display within the three-dimensional environment; and
provide the three-dimensional environment, with the representation, on the display.

14. The processor of claim 10, wherein the one or more probabilities are computed using one or more K-nearest neighbor algorithms.

15. The processor of claim 10, wherein the medical device data input includes identification information for a location associated with a device used to acquire the medical device data input.

16. A computer-implemented method, comprising:
generating one or more visualizations representative of data acquired by a functional lumen imaging probe (FLIP), the one or more visualizations corresponding to a region identified by a user including a start time index, an end time index, and one or more sensor indexes;
determining, from the data, a plurality of metrics to categorize an esophageal motility disorder, the plurality of metrics including a first metric associated with contractile response and a second metric associated with an esophagogastric junction (EGJ) opening;
determining a first plurality of sub-metrics for the first metric corresponding to a classification of a contraction pattern;
determining a second plurality of sub-metrics for the second metric corresponding to an EGJ distensibility index and a categorization for a maximum EGJ opening;
determining, using one or more machine learning algorithms, one or more probabilities of obstruction at the EGJ based, at least in part, on the first metric and the second metric;
determining, based on the one or more probabilities of obstruction and at least one of the first metric or the second metric, a treatment for a patient associated with the data, wherein the treatment is selected based on a likelihood of decreasing the one or more probabilities of obstruction;
providing, on a display and responsive to a request in real or near-real time during a procedure associated with collection of the data, at least one of the one or more visualizations, the one or more probabilities, and at least a portion of the plurality of metrics;
generating an indication of a mechanical cause associated with the esophageal motility disorder;
determining based, at least in part, on the mechanical cause and the one or more probabilities, a treatment path corresponding to the mechanical cause; and
determining, responsive to second data after treatment, selected according to the treatment path, for the mechanical cause, an assessment of treatment success based, at least in part, on updated one or more probabilities of obstruction.

17. The computer-implemented method of claim 16, wherein the one or more visualizations correspond to a multi-dimensional latent space representation presented as a three-dimensional environment.

18. The computer-implemented method of claim 17, wherein the multi-dimensional latent space representation includes a grouped set of datapoints from historical data corresponding to the one or more diagnostic groups.

19. The computer-implemented method of claim 17, further comprising:
receiving a second medical device data input;
determining, using at least a portion of the second medical device data input, a representation for display within the three-dimensional environment; and
providing the three-dimensional environment, with the representation, on the display.

20. The computer-implemented method of claim 16, wherein the one or more probabilities of obstruction are computed using one or more K-nearest neighbor algorithms.
